(19) United States
(12) Patent Application Publication
(10) Pub. No.: US 2022/0283147 A1
(43) Pub. Date: Sep. 8, 2022

(54) METHOD AND FLOW CYTOMETER FOR EXAMINING A HUMAN OR ANIMAL CELL SPECIMEN, AND COMPUTER PROGRAM PRODUCT

(71) Applicant: TOPOSNOMOS LTD., Munchen (DE)

(72) Inventor: Walter Schubert, Leipzig (DE)

(21) Appl. No.: 17/633,423

(22) PCT Filed: Aug. 5, 2020
(86) PCT No.: PCT/EP2020/071996

§ 371 Date: Feb. 7, 2022

(30) Foreign Application Priority Data
Aug. 7, 2019 (DE) 10 2019 1211344.4

(51) Int. Cl. G01N 33/50; G01N 33/58; G01N 15/14

(52) U.S. Cl. CPC G01N 33/5091; G01N 33/582; G01N 15/1434; G01N 2015/1006

(57) ABSTRACT

The invention relates to a method for examining a human or animal cell specimen, comprising the steps: a) providing at least one combinatorial cluster (CMP) which is characteristic of a disease, the CMP characterizing the colocalization and/or anticolocalization of multiple biological features in a voxel; b) determining N tags which are sufficient to determine CMP; c) bringing the cell specimen into contact with the N tags; d) measuring the cell specimen by means of flow cytometry to obtain flow cytometric data; and e) using the flow-cytometric data to check whether the CMP occurs in the cell. The invention further relates to a flow cytometer (10).

The invention relates to a method and to a flow cytometer for examining a human or animal cell specimen. Furthermore, the invention relates to a computer program product.

Multi-epitope ligand cartography (MELK) and ICM methods, respectively, (multi-epitope ligand cartography/imaging cycler microscopy), represent the basic technology for the detection of large molecular networks in cells or tissues with virtually unlimited combinatorial molecular discriminability per data point (“power of combinatorial molecular discrimination per date point”, PCMD). The ICM method and the MELK robot system, respectively, are known as such for example from the documents U.S. Pat. No. 6,150,173, DE 197 09 348 A1, EP 0 810 428 A1, DE 100 43 470 A1 and WO 02/21137 A2. The high PCMD is based on the fact that with the aid of the ICM, for example of k=100 different proteins, images can be captured and processed with the aid of a 16 bit CCD camera (2\hat{ }16=65536 values per data point or pixel), whereby 65536 k values can be ascertained per data point. Therein, each data point corresponds to a certain voxel, that is a grid point (“image” point, data element), in a three-dimensional grid of the examined cell specimen. The dimensions of an individual voxel substantially depend on the optical characteristics of the elements of the used ICM device (camera, lenses etc.). In this manner, so-called protein clusters can for example be detected, which are a combinatorial molecular phenotype (CMP), which describes colocalized and anticolocalized, respectively, CDs (clusters of differentiation, discrimination groups), proteins and/or other molecules or target structures at a certain location of a cell. Accordingly, groups of CMPs represent regions of colocalized and/or anticolocalized CDs, proteins and/or other molecules in a cell or a cell complex of the cell specimen. For example, a CMP can be indicated in the form of a binary code, wherein it is possible to encode via each digit of the binary number (i.e. via each “bit”) if a predetermined CD/protein/molecule at a certain location and/or in a concentration exceeding a certain limit value (e. g. L, 1 or True) occurs in the cell. In the other case, that is if the predetermined CD/protein/molecule does not occur at this location of the cell or only in a concentration below a limit value, a varying encoding (e.g. A, 0 or False) is used. Similarly, it can occur that certain CDs/proteins/molecules are only sometimes colocalized with one or more other CDs/proteins/molecules and sometimes not colocalized. These CDs/proteins/molecules can for example be encoded with “wildcards” (e.g. W, *). Such ICM data can be used for identification of patterns and targets specific to disease and can therefore be lifesaving. Therein, the functional protein pattern and the entirety of the molecular networks of the cells and tissues, respectively, which are identifiable with the aid of the ICM, are also referred to as toponome referring to the genome.

The disadvantage in the known ICM methods and devices is in that the examination of a cell specimen is comparatively lengthy according to number of the CDs/proteins/molecules to be examined and therefore currently cannot yet be employed as a high-throughput method for example for the clinical care. Therefore, cellular toponomes specific to disease of many patients cannot be measured per day and laboratory with ICM up to now.

It is the object of the present invention to allow a faster diagnosis of a human or animal cell specimen.

According to the invention, the objects are solved by a method with the features of claim 1, by a flow cytometer according to claim 14 as well as by a computer program product according to claim 15. Advantageous configurations with convenient developments of the invention are specified in the respective dependent claims, wherein advantageous configurations of each inventive aspect are to be regarded as advantageous configurations of the respectively other inventive aspects.

A first aspect of the invention relates to a method for examining a human or animal cell specimen. According to the invention, the method includes the steps of a) providing at least one combinatorial cluster (CMP) characteristic of a disease, wherein the CMP characterizes the colocalization and/or anticolocalization of multiple biological features in a voxel, b) determining N tags, which are sufficient for determining the CMP, c) contacting the cell specimen with the N tags, d) measuring the cell specimen by means of flow cytometry while obtaining flow-cytometric data and e) based on the flow-cytometric data, checking if the CMP occurs in the cell specimen.

Thus, the method according to the invention describes a multi-stage procedure. In a first step a), at least one combinatorial cluster (CMP) specific to disease is provided, which has for example been previously determined by ICM. The at least one CMP can for example be provided in the form of a PCMD code (1/0 code) or be converted into such one. Subsequently, the number N is determined in step b), which expresses as a natural number, how many tags are sufficient for determining the provided CMP (e.g. CMP=[CD16:1, CD8:1, NeuN:0, Bax:0, Bcl2:0] requires 5 markers for CD16, CD8, NeuN, Bax and Bcl2). A marking agent for the concerned target structure is understood by a tag. Usually, antibodies are used hereto, wherein other types of tags (magnetic beads, quantum dots etc.) can also be provided. If the CMP contains wildcard information, thus target structures, which can be, but do not have to be present, the tag required for identification of the concerned target structure can optionally be omitted. Subsequently, the cell specimen is contacted with the N determined tags in step c) and measured by means of flow cytometry while obtaining flow-cytometric data in step d). In the flow cytometry, the cells of the cell specimen are individually passed past an electrical voltage or a laser beam with high velocity. Depending on the tags bound to the cells, different effects are generated, from which the flow-cytometric data can be derived. Optionally, one or more further characteristics of the cells like size, shape, type etc. can additionally be determined and stored in the flow-cytometric data. It is understood that the tags are preferably generally selected such that they are identifiable with the respectively used flow-cytometric approach. Finally, it is checked if the CMP occurs in the cell specimen, based on the flow-cytometric data in step e). If the searched CMP occurs in the cell specimen, it is to be assumed that the cell specimen originates from a diseased patient, who suffers that disease, to which the CMP is characteristic. With the aid of the method according to the invention, a substantially higher throughput can be achieved compared to known ICM approaches since current flow cytometers can measure a high number of cells per second. Accordingly, more cell specimens can be examined in shorter time. Within the scope of the flow-cytometric measurement, each individual cell can formally be treated as a voxel, which is examined for the presence of the at least one CMP. Compared to usual ICM approaches, a reduction of the information density is associated therewith, since the dimensions of an ICM voxel are quasi scaled to the dimension of a cell or in that multiple CMPs and/or voxels are summed up or combined to a cell-specific value. However, this resolution reduced with respect to the ICM results has proven to be sufficient in practice to reliably identify predetermined CMPs specific to disease in flow-cytometric manner in individual cells. Therefore, a fast toponome diagnosis specific to disease is allowed for the first time by the method according to the invention, which correspondingly allows therapies specific to patient. Besides a use for diagnosis, the method according to the invention can of course also be used for further purposes, for example for the therapy monitoring of patients.

Therein, it has proven to be advantageous if a body fluid, in particular blood and/or blood plasma, and/or a tissue specimen of the patient are provided as the cell specimen. Hereby, the cell specimen can be optimally selected depending on the disease to be treated.

Further advantages arise in that the at least one CMP is determined based on a database and/or by means of a multi-epitope ligand cartography (MELK) and ICM method, respectively, (multi-epitope ligand cartography/imaging cycler microscopy) and/or by means of a MELK robot system. For diseases, in which the characteristic CMP or the characteristic group of CMPs is already known, it can be sufficient to provide the CMP or CMPs from a corresponding medical database, in which the CMP is stored. Alternatively or additionally, it can be provided that the CMP or CMPs characteristic of the disease is or are determined with the aid of a multi-epitope ligand cartography (MELK) or ICM (imaging cycler microscopy) method and/or by means of a MELK robot system.

In a further configuration of the invention, it is provided that the disease is a tumor disease and/or an inflammatory disease and/or an autoimmune disease and/or a disease, which resembles autoimmune phenomena, and/or a disease, which includes pathological homing processes. Such diseases have a particularly high extent of cellular organization and can be correspondingly reliably identified within the scope of the present invention. Diseases resembling autoimmune phenomena are particularly characterized in that mononuclear cells with features of peripheral T cells penetrate into organs, where they interact with the organ-specific cells, for example compress them, but do not cytotoxically attack them. Such phenomena were discovered and presented by means of ICM for example in the so-called postmitotic system (skeletal muscle, 1^{st} motoneuron). Solely this interaction is a possible cause for chronic, autoimmune organ damages.

In a further advantageous configuration of the invention, it is provided that the N tags and the cell specimen are each divided into at least two groups and measured. Hereby, possible cross reactions between certain tags can be advantageously avoided, whereby the measurement quality is increased. Moreover, more tags can be measured in this manner than it would normally be possible with the flow-cytometric approach and the used flow cytometer, respectively. Within the scope of the present disclosure, 2, 3, 4, 5, 6, 7, 8, 9, 10 or more groups are to be understood by at least two groups, which each can contain one tag or 2, 3, 4, 5, 6, 7, 8, 9, 10 or more tags. The number of groups as well as the division of the tags to be measured to the individual groups substantially depends on how many channels or detectors the flow cytometer used for the measurement comprises and which signals of the individual tags have to be detected. If the flow cytometer for example provides 21 fluorescence channels, those tags, which are required for measuring a 42-digit CMP, can be divided into two or more groups. Subsequently, the flow-cytometric data of the individual groups can then again be combined to a common dataset.

Further advantages arise in that the cell specimen is divided into at least one first partial specimen, which comprises the at least one CMP, and a second partial specimen, which does not comprise the at least one CMP, after measurement. Hereby, suspicious cells can be separated from unsuspicious cells and optionally be subjected to further examinations. For example, the division can be effected via a cell sorter, which is arranged downstream of the flow cytometer and divides the cells into two or more fractions depending on the determined flow-cytometric data.

In a further configuration, it is provided that the disease is amyotrophic lateral sclerosis (ALS) if the cell specimen is a blood specimen and the CMP occurring in the cell specimen includes one or more from the group of CD16, CD8, NeuN, Bax, Bcl2, CD11b, CD138, CD16A, CD29, CD2, CD45RA, CD49d, CD54, CD56, CD57, CD58, CD62L, CD3, HLADR, immunoglobulin G, MHCII, MHCI, SIRT1, RAC1, BMX, GAK, JNK2, MAPKK6, OTUB2, PRKAR2A, SMAD2, SMAD4 and STAP2. The amyotrophic lateral sclerosis (ALS) is based on an unstoppably and rapidly progressing irreversible paralysis of the musculature since certain neural cells, which control the musculature, degenerate. Up to now, over 50 clinical treatment studies were performed, but the therapy concepts of which have predominantly proven to be ineffective. With the aid of the method according to the invention, it is possible for the first time to examine the cell specimen for the presence of these pathogenic ALS-specific cells in high throughput. If the ALS-specific cells are detected, the patients can be treated already in very early stages of the disease. For example, the ALS-specific cells can be extracorporeally removed or inhibited with the aid of a photopheresis treatment, whereby the disease progression can be stopped or at least substantially slowed down. The pathogenic constituents responsible for the ALS are aberrant T lymphocytes, which also express the CD16 receptor complex besides the CD8 receptor. Therefore, they are characterized by CMPs, which contain CD8 and/or CD16 as lead proteins. The lead proteins can be colocalized and anticolocalized, respectively, with the further specified CDs and proteins. Accordingly, it has proven to be advantageous if a tag used for examining the cell specimen is an antibody and/or ligand, which binds one, two, three, four, five or more from the group of CD16, CD8, STTP1, NeuN, Bax, Bcl2, CD11 b, CD138, CD16A, CD29, CD2, CD45RA, CD49d, CD54, CD56, CD57, CD58, CD62L, CD3, HLADR, immunoglobulin G, MHCII, MHCI, SIRT1, RAC1, BMX, GAK, JNK2, MAPKK6, OTUB2, PRKAR2A, SMAD2, SMAD4 and STAP2.

In other words, it is provided that an antibody and/or ligand is used as the tag, which is mono-, bi-, tri-, quad-, pent-, hex-, hept-, oct-, enn- or multi-specifically formed. The mentioned proteins form over-individual and individual clusters in ALS-specific cells in different combinations and can therefore be precisely detected and subsequently optionally extracorporeally removed and/or therapeutically crosslinked and thereby blocked, whereby the ALS-specific cells lose their functionality. Hereby, a particularly specific treatment of ALS, thereby low in side effects or even free of side effects is allowed. Further advantages arise if at least one tag binds at least CD16, CD8 and STTP1. Herein, STTP1 denotes the optionally patient-specific signal protein (signal transduction protein 1, e. g. kinase), which mediates the signal cascade, which extends from the cell surface to the nucleus, and couples together with the module “CD16a and CD8” on the surface of the cell, where it is colocalized with CD16a and CD8. Furthermore, it can be provided that STTP1 is at least one protein from the group of RAC1, STAP2 and SMAD2. Further advantages arise if at least one tag includes an antibody and/or ligand, which is recombinantly, humanely or de novo produced.

Furthermore, it can be provided that the disease is prostate cancer if the cell specimen is prostate tissue and the at least one CMP includes one or more from the group of CD26 and CD29. Hereby, it is possible for the first time to examine cell specimens for the presence of CMPs indicating prostate cancer in high-throughput method. In this case too, it is reasonable to treat patients, in whom the mentioned CMP was identified in the cell specimen, as fast as possible. Therein, the invention is based on the realization that prostate cancer is characterized by CMPs, which contain CD26 and/or CD26 as lead proteins, which are optionally colocalized and anticolocalized, respectively, with further CDs and proteins, respectively.

Furthermore, it can be provided that the disease is a cutaneous lymphoma if the cell specimen is a skin specimen and the at least one CMP includes one or more from the group of HLA-DQ, CD2, CD3, CD4, CD7, CD8, CD10, CD13, CD18, CD18, CD26, CD29, CD36, CD44, CD45, CD49f, CD54, CD56, CD57, CD58, CD62L, CD71, CD80 and HLA-DR. Thereby, it is possible for the first time to examine cell specimens for the presence of CMPs in the high-throughput method, which are characteristic of a cutaneous lymphoma. Therein, the invention is based on the realization that cutaneous lymphomas are characterized by CMPs, which in particular contain HLA-DQ as the lead protein, which is colocalized and anticolocalized, respectively, with one or more of the mentioned CDs and proteins, respectively.

In a further advantageous configuration of the invention, it is provided that at least one gate is first defined before checking the flow-cytometric data, by means of which a subset of the cell specimen and/or of the data is selected for the check. Hereby, detailed analyses of certain subsets can be performed. For example, a subpopulation of the measured cells can be selected for further analysis. The subpopulation of cells within a gate can for example be graphically highlighted and/or be displayed together with information from other channels or data sources. The definition of gates offers high flexibility in the flow cytometry and allows a resolution up to individual cells for each channel of the used flow cytometer. Similarly, it is possible to define, to “stack” and/or to combine multiple gates. This allows a correspondingly flexible and highly precise evaluation of the determined flow-cytometric data.

Particularly many cell specimens can be measured in a time as short as possible in that the measurement of the cell specimen in high throughput is effected with at least 5000 cells, thus for example 5000, 6000, 7000, 8000, 9000, 10000 or more cells per second.

Further advantages arise in that constituents of the cell specimen, which comprise the at least one CMP, are classified as pathogenic and/or redundant. Then, the concerned cells can be specifically isolated with the aid of methods known per se and be further used for diverse purposes, e. g. cloning, extracorporeal expansion, retransfer into tissues and blood, respectively, therapeutic applications etc.

In a further advantageous configuration of the invention, it is provided that constituents of the cell specimen classified as pathogenic and/or redundant are extracorporeally removed, in particular by means of an apheresis method and/or by means of an apheresis device. This allows the isolation of cells specific to disease from the blood circulation, which for example immigrate to organs as autoimmune cells or aberrant cells of the immune system, where they specifically destruct tissue structures. In the latter case, e. g. the isolation of such cells from the blood circulation by a therapeutic apheresis, in particular by photopheresis, would be a very purposeful measure with the aim of the stop of the disease progression since the cells specific to disease can be exactly predefined by highly dimensional toponome mapping via their CMPs specific to disease. Alternatively or additionally, the aberrant cells can be specifically removed from the circulation via isolation based on antibody. Via consecutive monitoring of the blood with the aid of the method according to the invention, the efficiency of the therapy can be controlled. A further advantage is in the use of such cells for the development of medicaments for selective blocking/elimination of such cells or, in case of stem cells, for reinfusion into the organism (e. g. therapeutic organ regeneration or tumor therapy) or for use for the development of diagnostics or for the therapy of the amyotrophic lateral sclerosis (ALS) or other diseases by removal of pathogenic cells from the blood circulation to prevent the biological mechanism thereof, the invasion into the motoneuron system and the neurotoxic damaging mechanisms thereof in the case of ALS.

A second aspect of the invention relates to a flow cytometer comprising a control device, which can be coupled to a database, to determine at least one combinatorial cluster (CMP) characteristic of a disease, wherein the CMP characterizes the colocalization and/or anticolocalization of multiple biological features in a voxel, a determination device for determining N tags, which are sufficient for determining the CMP, a dosing device, by means of which the N tags can be contacted with a cell specimen, a measurement device for measuring the cell specimen by means of flow cytometry while obtaining flow-cytometric data, and a checking device, by means of which it can be checked if the CMP occurs in the cell specimen, based on the flow-cytometric data. With the aid of the flow cytometer according to the invention, a substantially higher throughput can be achieved compared to known ICM systems since a high number of cells can be measured per second. Accordingly, more cell specimens can be examined in shorter time. By the flow cytometer according to the invention, therefore, a fast toponome diagnosis specific to disease is allowed for the first time, which correspondingly allows therapies specific to patient. Besides a use for diagnosis, the flow cytometer according to the invention can of course also be used for further purposes, for example for the therapy monitoring of patients. Further features and advantages are apparent from the description of the first inventive aspect.

A third aspect of the invention relates to a computer program product, which can be loaded into a memory of a control device of a flow cytometer according to the second inventive aspect, wherein the computer program product comprises program means to execute the steps of the method according to the first inventive aspect when the program is executed by a processor device of the control device. The advantages achievable hereby can be taken from the descriptions of the preceding inventive aspects. In a further aspect, an electronically readable data carrier with electronically readable control information stored thereon can be present, which includes at least one described computer program product and is configured such that it performs a described method according to the first inventive aspect upon use of the data carrier in a control device of a flow cytometer.

Further features of the invention are apparent from the claims and the embodiments. The features and feature combinations mentioned above in the description as well as the features and feature combinations mentioned below in the embodiments and/or shown alone are usable not only in the respectively specified combination, but also in other combinations or alone without departing from the scope of the invention. Thus, implementations are also to be considered as encompassed and disclosed by the invention, which are not explicitly shown and explained in the embodiments, but arise from and can be generated by separated feature combinations from the explained implementations. Implementations and feature combinations are also to be considered as disclosed, which thus do not comprise all of the features of an originally formulated independent claims. There shows:

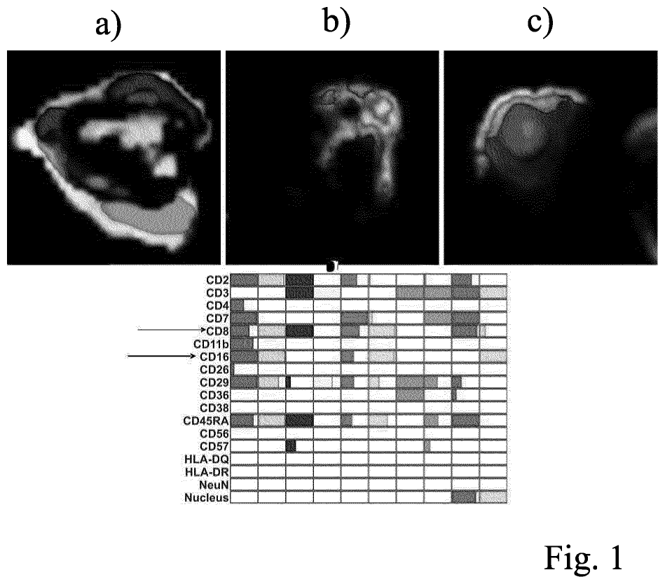

FIG. 1 an ICM documentation of an extracorporeal photopheresis treatment of a patient with the effect thereof on an ALS-specific cell;

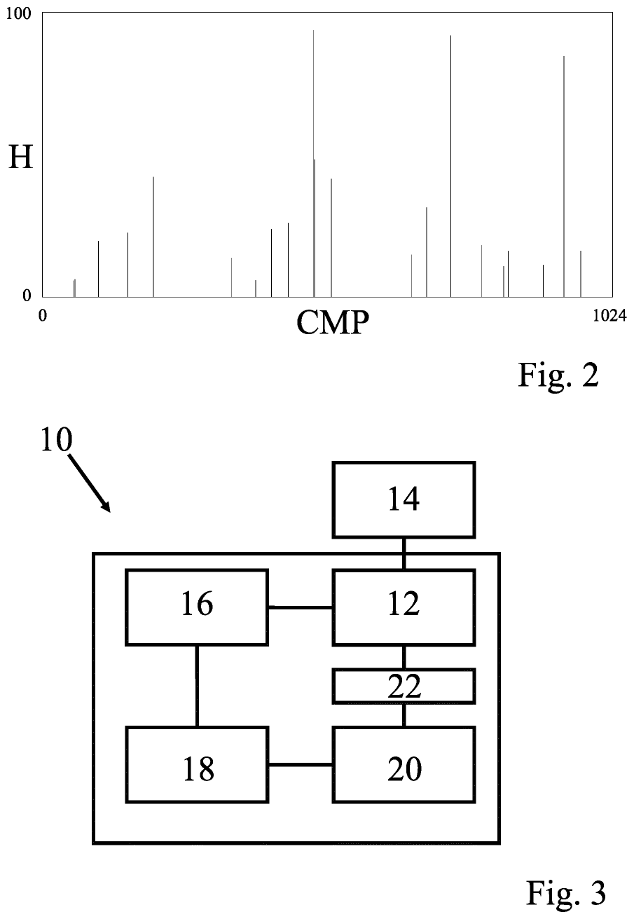

FIG. 2 a diagram of a toponome fingerprint; and

FIG. 3 a schematic representation of a flow cytometer according to the invention.

The imaging cycler microscopy (ICM) is the basic technology for the detection of large molecular networks in intact cells or tissues with virtually unlimited combinatorial molecular discriminability per data point (“power of combinatorial molecular discrimination per date point”, PCMD). High PCMD was shown for up to 100 proteins or more (with k=100 different proteins and 65536 values per pixel output of a 16 bit CCD camera in an ICM, for example 65536 k values per voxel result). Such and similar ICM data is specific to disease and can be lifesaving, but ICM methods currently cannot yet be employed as high-throughput methods for the clinical care. The method according to the invention avoids this disadvantage by a multi-stage method flow. In a first step, CMPs specific to disease are provided, which for example have been detected on cells specific to disease by means of ICM and the spatial PCMD code of which is expressed in the form of combinatorial clusters (CMPs). These CMPs, which define the colocalization and/or anticolocalization of a plurality of proteins/molecules/target structures in a voxel, can be expressed as a sum code per cell. In a 1/0 or true/false notation, such a code can for example be expressed as a binary number 0000001001, wherein each digit of the binary number corresponds to a predefined protein/molecule and a predefined target structure, respectively. Of course, other notations and data encoding types (e. g. decimal notation, hexadecimal notation, xml, json etc.) can generally also be used.

In that one plots all of the possible CMPs for a given tag number (e. g. for 10 tags 0000000000, 0000000001, 0000000010, 0000000011, 0000000100, 0000000101, 0000000110, 0000000111, 0000001000, 0000001001, 0000001010 etc. up to 111111111 or in decimal notation 0, 1, 2, 3, 4, 5, 6, 7, 8, 9, 10 etc. up to 1024) on an abscissa axis (x-axis) and the frequency H of the concerned CMP found in the cell specimen on the ordinate axis (y-axis), diagrams can be created, which are characteristic of the toponome of the measured cell specimen and can be used for “fingerprinting” of diseases. As already mentioned, each binary digit stands for the presence (1) or non-presence (0) of a predetermined target structure (e. g. a CD) in a predetermined voxel. Similarly, such “toponome fingerprints” can be used for identifying potential medical problems by comparison to a “toponome fingerprint” of a cell specimen classified as “healthy”. FIG. 2 exemplarily shows such a diagram of a “toponome fingerprint”, from which the CMPs characteristic of a certain disease and their respective frequency are apparent. The creation and/or representation and/or use of such toponome fingerprint diagrams represent a separate inventive aspect.

This conversion and representation has proven to be helpful to fast and accurately identify cells specific to disease in blood to therapeutically deplete them as a result. The highly dimensional ICM-PCMD code can then be transformed in a pan-cellular I/O code and be used for the analysis of a cell specimen within the scope of a high-throughput measurement in a flow cytometer. This allows to measure the cellular toponome specific to disease of many patients per day and laboratory and to transfer it into a specific depletion therapy. Thereby, a toponome therapy specific to disease of for example cellular autoimmune processes specific to organ is allowed for the first time.

The imaging cycler microscopy (ICM) allows to spatially resolve large molecular networks—so-called toponomes—in the morphologically intact tissue. Therein, a virtually arbitrarily high combinatorial molecular resolution per data point or voxel (power of combinatorial molecular discrimination per data point, PCMD) is achieved.

For example, in a basal lamina (BL) of the human skin, ICM can both structurally and functionally discriminate 6 layers within the BL with a diameter of 125 nm (Schubert, W. (2018): A Platform for Parameter Unlimited Molecular Geometry Imaging Obviously Enabling Life Saving Measures in ALS. Advances in Pure Mathematics, 8, 321-334. https://doi.org/10.4236/apm.2018.83017). Therefore, ICM allows high structural resolution and functional resolution at the same time at one and the same location. Remarkably, these ICM layers are delimited from each other by sharp separating lines. These separating lines, which are here referred to as Riemann-Zeta-like function (RZLF), obviously serve to the fact that the different functions of the 6 layers can be kept biologically separated from each other. Based on the correlations identified by means of ICM, mononuclear cells of the blood were identified with ICM in the sporadic form of the ALS, which immigrate into the pyramidal tract (1st motoneuron), where they compress motoric axons (so-called axotomy-competent cells (ACC)). With the aid of the ICM, thus, the mechanisms of the disease ALS could be directly visualized in the affected tissue. This data implies that the detection of ACC in the blood indicates an active and finally lethal axotomy process such that an absolute indication for immediate therapeutic depletion of these cells is given.

FIG. 1 shows an ACC measured by ICM (post-mortem) in image a), an ACC of an ALS patient measured by ICM in image b) and an ACC cell of the patient measured by ICM after he was treated with extracorporeal photopheresis (ECP), in image c). The table below FIG. 1a)-c) shows the definition of different CMPs, wherein each CMP has been color-coded. Therein, CMPs, in which CD8 and CD16 are colocalized, are specific to disease for the ALS.

As one sees in FIG. 1, the ACC were subcellularly severely damaged by the therapeutic treatment with photopheresis. This damage could be evidenced by ICM over a longer period of time in multiple examinations. Parallel in time, the clinical disease signs of the patient regressed. These observations are consistent with collected data, which shows that the number of the ACC per liter of blood correlates with the progression speed of the disease.

The above described facts imply that the depletion therapy of the ACC is an effective lifesaving measure in case of ALS and should be available for all ALS patients, in particular since there is no alternative as a lifesaving therapy. However, a problem is in that the ALS often could only be diagnosed in an advanced stage heretofore.

In order to reach many patients with above mentioned indication and other similar indications in case of organ-specific autoimmune processes or autoimmune-similar processes, e.g. Hashimoto Thyroiditis or multiple sclerosis, analogously to the procedure in case of ALS, the following method is performed in case of these and similar indications or suspected cases:

1. If CMPs specific to disease are not yet known, ICM diagnostics is performed on mononuclear cells (MNZ) of the blood:
1) MNZ are isolated from whole blood;
2) Isolated MNZ are shortly air-dried and then deep-frozen via isopentane in liquid nitrogen and stored deep-frozen until use;
3) an ICM measurement is performed as follows:
   The MNZ specimen is rehydrated with PBS at room temperature and then placed on the object table for measurements in the ICM and cyclically marked with a marker library, which includes N tags. Therein, N can be up to 100 or more. The ICM marker combinations are determined in that the tag combinations present in the MNZ are determined for each tag. The tag combinations can then be expressed as CMP in the form of an I/O encoding. All of the markers can then be plotted in a diagram for illustration, wherein the colocalization and anticolocalization code (I/O) is plotted on the x-axis of a diagram and the respective frequency is plotted on the y-axis. Such a diagram is exemplarily shown in FIG. 2 as already mentioned.
4) As soon as the finding under (3) is present, the search for the MNZ specific to disease with the same marker library is transferred to a flow cytometer, which for example comprises 6 lasers and 21 fluorescence channels, for the same patient: Here, each individual MNZ is regarded as a point, which expresses a given 1/0 code specific to disease. This MNZ form can then be quantified in high throughput per patient before, during and after the ECP therapy and be subcellularly exactly measured by intermitting ICM.
5. The advantage of this approach is in that the ECP allows an exact toponome measurement in a two-step method in high throughput by coupling of ICM/flow cytometry for the first time. Thereby, it is possible to toponomically exactly measure all of the relevant patient groups.

If the CMP data is already known, it can for example be provided from a database, optionally transformed and used for the flow-cytometric measurement.

FIG. 3 shows a schematic representation of a flow cytometer 10 according to the invention. The flow cytometer 10 includes a control device 12, which is wirelessly or wired coupled to a presently external database 14 for data exchange to determine at least one combinatorial cluster (CMP) characteristic of a disease, wherein the CMP characterizes the colocalization and/or anticolocalization of multiple biological features in a voxel. Among other things, the control device 12 usually comprises a memory and a processor device to execute software. Basically, the database 14 can also be a part of the flow cytometer 10. Furthermore, the flow cytometer 10 includes a determination device 16 for determining N tags, which are sufficient for determining the CMP. Generally, the determination device 16 can be omitted if the required information about the required N tags can be otherwise provided. Furthermore, the flow cytometer 10 comprises a dosing device 18, by means of which the N tags can be contacted with a cell specimen (not shown), as well as a measurement device 20 for measuring the cell specimen by means of flow cytometry while obtaining flow-cytometric data. Generally, the dosing device 18 can also be omitted if the cell specimen has already been contacted with the N tags outside of the flow cytometer 10. Finally, the flow cytometer 10 includes a checking device 22, by means of which it is checked if the CMP occurs in the cell specimen, based on the flow-cytometric data. If the CMP occurs in the cell specimen, it is to be assumed that the patient suffers a disease, of which the found CMP is characteristic. This finding allows an early diagnosis and therapy derivable therefrom.

The parameter values specified in the documents for definition of process and measurement conditions for characterizing specific characteristics of the inventive subject matter are to be regarded as encompassed by the scope of the invention also within the scope of deviations—for example due to measurement errors, system errors, weighing errors, DIN tolerances and the like.

1-15. (canceled)

16. A method for examining a human or animal cell specimen, comprising the steps of:
a) providing at least one combinatorial cluster (CMP) characteristic of a disease, wherein the CMP characterizes the colocalization and/or anticolocalization of multiple biological features in a voxel;
b) determining N tags, which are sufficient for determining the CMP;
c) contacting the cell specimen with the N tags;
d) measuring the cell specimen by means of flow cytometry while obtaining flow-cytometric data; and
e) based on the flow-cytometric data, checking if the CMP occurs in the cell specimen.

17. The method according to claim 16, wherein a body fluid blood and/or blood plasma, and/or mononuclear cells of the blood and/or a tissue specimen are provided as the cell specimen.

18. The method according to claim 16, wherein the at least one CMP is determined based on a database and/or by means of a multi-epitope ligand cartography (MELK) and/or ICM (imaging cycler microscopy) method and/or by means of a MELK robot system.

19. The method according to claim 16, wherein the disease is a tumor disease and/or an inflammatory disease and/or an autoimmune disease and/or a disease resembling AI phenomena and/or a disease, which includes pathological homing processes.

20. The method according to claim 1, wherein the N tags and the cell specimen are each divided into at least two groups and measured.

21. The method according to claim 16, wherein the cell specimen is divided into at least one first partial specimen, which comprises the at least one CMP, and a second partial specimen, which does not comprise the at least one CMP, after measurement.

22. The method according to claim 16, wherein the disease is amyotrophic lateral sclerosis (ALS) if the cell specimen is a blood specimen and the CMP occurring in the cell specimen includes one or more from the group of CD16, CD8, NeuN, Bax, Bcl2, CD11 b, CD138, CD16A, CD29, CD2, CD45RA, CD49d, CD54, CD56, CD57, CD58, CD62L, CD3, HLADR, immunoglobulin G, MHCII, MHCI, SIRT1, RAC1, BMX, GAK, JNK2, MAPKK6, OTUB2, PRKAR2A, SMAD2, SMAD4 and STAP2.

23. The method according to claim 16, wherein the disease is prostate cancer if the cell specimen is prostate tissue and the at least one CMP includes one or more from the group of CD26 and CD29.

24. The method according to claim 16, wherein the disease is a cutaneous lymphoma if the cell specimen is a skin specimen and the at least one CMP includes one or more from the group of HLA-DQ, CD2, CD3, CD4, CD7, CD8, CD10, CD13, CD18, CD18, CD26, CD29, CD36, CD44, CD45, CD49f, CD54, CD56, CD57, CD58, CD62L, CD71, CD80 and HLA-DR.

25. The method according to claim 16, wherein before examining the flow-cytometric data, at least one gate is first defined, by means of which a subset of the cell specimen and/or of the data for the check is selected.

26. The method according to claim 16, wherein the measurement of the cell specimen is effected in high throughput with at least 5000 cells per second.

27. The method according to claim 16, wherein constituents of the cell specimen, which comprise the at least one CMP, are classified as pathogenic and/or redundant.

28. The method according to claim 27, wherein constituents of the cell specimen classified as pathogenic and/or redundant are extracorporeally removed, by means of an apheresis method and/or by means of an apheresis device.

29. A flow cytometer, comprising:
a control device, which can be coupled to a database, to determine at least one combinatorial cluster (CMP) characteristic of a disease, wherein the CMP characterizes the colocalization and/or anticolocalization of multiple biological features in a voxel;
a determination device for determining N tags, which are sufficient for determining the CMP;
a dosing device, by means of which the N tags can be contacted with a cell specimen;
a measurement device for measuring the cell specimen by means of flow cytometry while obtaining flow-cytometric data; and
a checking device, by means of which it can be checked if the CMP occurs in the cell specimen, based on the flow-cytometric data.

30. A computer program product, which can be loaded into a memory of a control device of a flow cytometer according to claim 29, wherein the computer program product comprises program means to execute the steps of the method when the program is executed by a processor device of the control device.
